(19) United States
(12) Patent Application Publication
(10) Pub. No.: US 2002/0010484 A1
(43) Pub. Date: Jan. 24, 2002

(54) Microkeratome spring deceleration device

(76) Inventors: Miguel Cabada, Winter Springs, FL (US); Lewis Harrold, Winter Springs, FL (US); George Kocar, Orlando, FL (US)

Correspondence Address: MANELLI DENISON & SELTER PLLC, 7th Floor, 2000 M Street, N.W., Washington, DC, 20036-3307, US

(21) Appl. No.: 09/906,109

(22) Filed: Jul. 17, 2001

Related U.S. Application Data
(60) Provisional application No. 60/219,609, filed on Jul. 21, 2000.

(51) Int. Cl. A61F 9/00

(52) U.S. Cl. 606/166

(57) ABSTRACT

A keratome includes a body configured to be movable during a stroke thereof with respect to a suction ring assembly. The body includes spring structure constructed and arranged to resiliently contact a portion of the suction ring assembly at an end of the stroke of the body.

This application is based on U.S. provisional Application No. 60/219,609, filed on Jul. 21, 2000, and claims the benefit thereof for priority purposes. This U.S. Provisional Application is expressly incorporated herein by reference.

### FIELD OF THE INVENTION

The invention generally relates to microkeratomes for use in laser eye surgery and, more particularly, to a microkeratome that includes a spring structure to ensure a soft stop at the end of the microkeratome's travel.

### BACKGROUND OF THE INVENTION

In performing the primary lamellar cut for laser assisted in situ keratomileusis, before ablation of corneal tissue, a microkeratome is moved with respect to a suction ring assembly which holds eye tissue. Movement of the microkeratome slices corneal tissue. A conventional microkeratome includes a body carrying fine gearing that intermeshes with a gearing on the suction ring assembly to cause movement of the microkeratome with respect to the suction ring assembly. As the body reaches the end of its stroke, the body contacts a portion of the suction ring assembly in manner that can be considered to be a hard stop. The slice is therefore stopped before cutting entirely through the corneal tissue thereby forming a flap of tissue.

In contacting the suction ring assembly in this manner, the integrity of the fine gears of the microkeratome can be compromised. In some instances, due to the microkeratome's contact with the suction ring assembly, the gears can even “lock-up”. Thus, the contact of the microkeratome at the end of its stroke in the conventional hard stop manner reduces the life and function of the microkeratome.

There is a need to provide a microkeratome that includes spring structure ensuring that a soft stop of the microkeratome occurs at the end of the microkeratome's travel.

### SUMMARY OF THE INVENTION

An object of the invention is to fulfill the need referred to above. In accordance with the principles of the present invention, this objective is achieved by providing a keratome having a body configured to be movable during a stroke thereof with respect to a suction ring assembly. The body includes spring structure constructed and arranged to resiliently contact a portion of the suction ring assembly at an end of the stroke of the body.

In accordance with another aspect of the invention, a method is provided for damping forces resulting from contact of a keratome with a fixed suction ring assembly at an end of a stroke of the keratome. The suction ring assembly includes a stop surface and the keratome has a body movable with respect to a suction ring assembly. The body includes a resilient stop surface. The method includes moving the body with respect to the suction ring assembly such that at the end of the stroke, the resilient stop surface of the body engages the stop surface of the suction ring assembly so as to dampen a contact force between the suction ring assembly and the body.

Other objects, features and characteristics of the present invention, as well as the methods of operation and the functions of the related elements of the structure, the combination of parts and economics of manufacture will become more apparent upon consideration of the following detailed description and appended claims with reference to the accompanying drawings, all of which form a part of this specification.

### BRIEF DESCRIPTION OF THE DRAWINGS

The invention will be better understood from the following detailed description of the preferred embodiments thereof, taken in conjunction with the accompanying drawings, wherein like reference numerals refer to like parts, in which:

FIG. 1 is a side view of a gear plate of a microkeratome provided in accordance with the principles of the present invention.

FIG. 2 is a perspective view of the gear plate of FIG. 1.

FIG. 3 is a perspective view of a microkeratome including the gear plate of FIG. 2, shown associated with a suction ring assembly.

FIGS. 4A and 4B are schematic views of other embodiments of spring structure of the invention.

### DETAILED DESCRIPTION OF THE PREFERRED EMBODIMENT

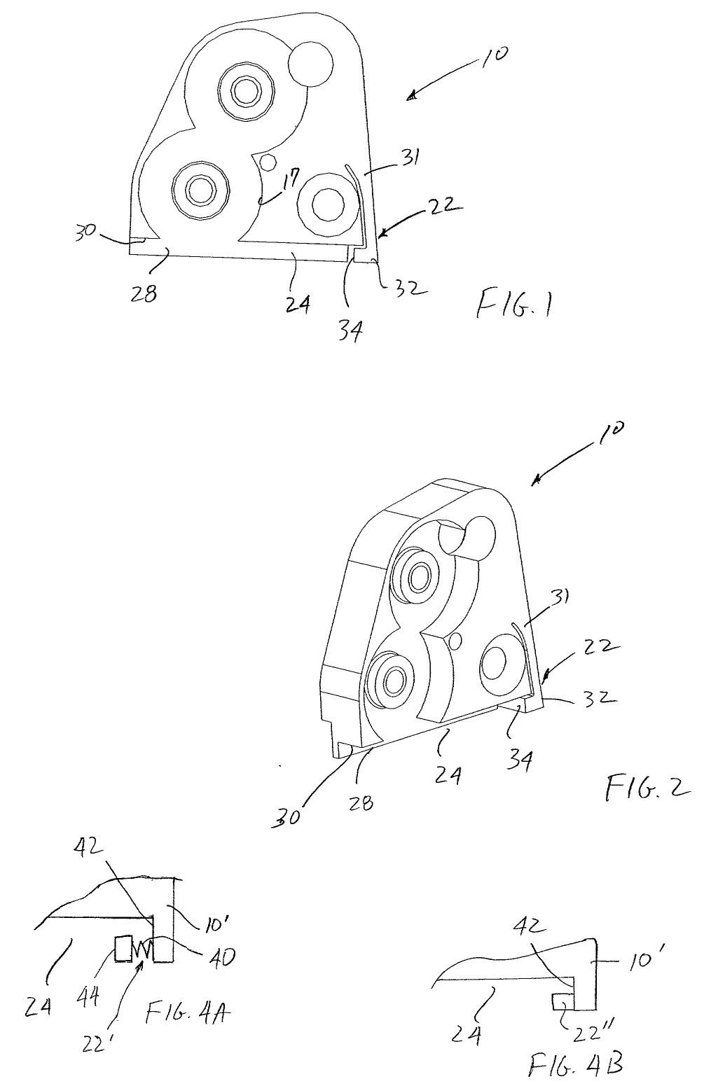

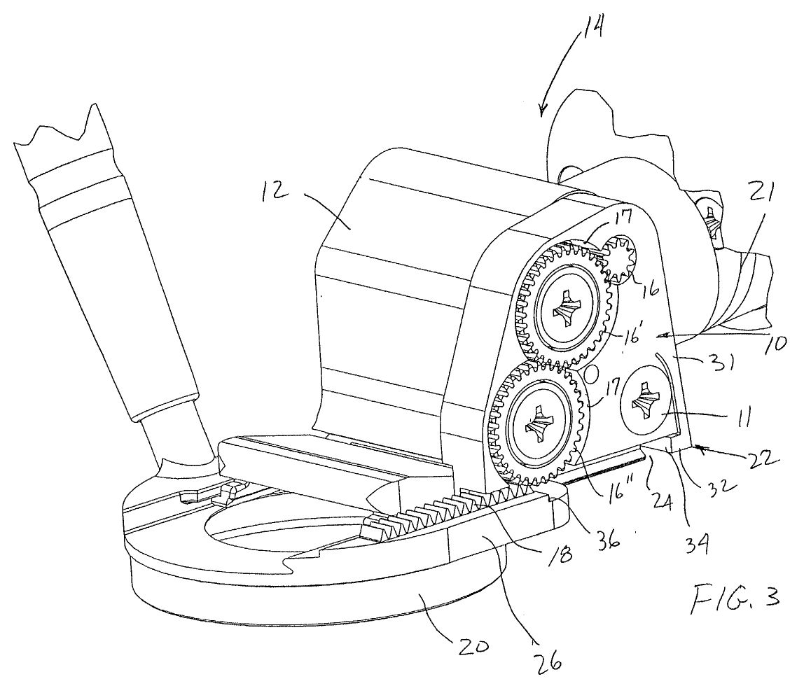

With reference to FIG. 1, a microkeratome gear plate, provided in accordance with the principles of the present invention is shown generally indicated at 10. As shown in FIG. 3, the gear plate 10 is attached by a fastener 11 to a body 12 of a microkeratome, generally indicated at 14. The gear plate 10 can be considered to be part of the body 12. In the conventional manner, the microkeratome 12 includes gears 16, 16′ and 16″ carried by the gear plate 10 within a gearing recess 17. The gear 16″ intermeshes with a gear 18 on a suction ring assembly 20 so that the body 12 of the microkeratome 14 can move during a stroke thereof with respect to the fixed suction ring assembly 20. In particular, the suction ring assembly 20 is configured to be fixed to an eyeball such that the microkeratome 14 can slice corneal tissue. A motor and transmission shaft assembly 21 causes movement of the body 12. The microkeratome 14 and suction ring assembly 20 may be of the type described in U.S. Pat. No. Re. 35,421, the entire content of which is hereby incorporated by reference into the present specification.

In order to prevent “hard stop” contact of the body 12 with a portion of the suction ring assembly 20 at the end of the stroke of the body 12, in accordance with the invention, the gear plate 10 of the body 12 includes spring structure, generally indicated at 22. In the illustrated embodiment, an open clearance channel 24 is defined at the bottom of the gear plate 10 such that the gear plate 10 can clear a gear support 26 of the suction ring assembly 20 during a stoke of the body 12. The channel 24 is defined by surfaces 28 and 30 of the gear plate 10. In the disclosed embodiment, the spring structure 22 is made integral with the gear plate 10 and is defined as a cantilever having a hinged end 31 and a free end 32 opposite the hinged end 31. Thus, the free end 32 can flex about the hinged end 31. The free end 32 extends into the channel 24 at an end of the gear plate 10 to define a resilient stop surface 34 of the gear plate 10.

With reference to FIG. 3, the gear support 26 of the suction ring assembly 20 includes a stop surface 36. Hence, when the body 12 reaches the end of its stroke, the resilient stop surface 34 of the spring structure 22 resiliently contacts the stop surface 36 of the gear support 26 of the suction ring assembly 20, thereby providing a “soft stop” of the body 12 with respect to the suction ring assembly 20. Due to the “soft stop”, a force created by contact between the body 12 and the suction ring assembly 20 is dampened. Thus, the mechanical integrity of the fine gears 16, 16′ and 16″ of the microkeratome 14 are preserved, thereby preserving the life and function of the microkeratome 14. The “soft stop” also eliminates lock-up of the microkeratome 14 at the end of its stroke that can occur in conventional microkeratomes employing a hard stop.

With reference to FIG. 4A, instead of defining the spring structure 22 as a cantilever, the spring structure 22′ may comprise a compression spring 40 between a surface 42 of the gear plate 10′ and a stop member 44. Thus, as the stop member 44 engages the stop surface 36 (FIG. 3) of the suction ring assembly 20, the spring 40 compresses to ensure a “soft stop” of the microkeratome. Alternatively, as shown in FIG. 4B, the spring structure 22″ can be any resilient material such as silicone pad coupled to surface 42. In either of these two embodiments, no hinged end is required.

The foregoing preferred embodiments have been shown and described for the purposes of illustrating the structural and functional principles of the present invention, as well as illustrating the methods of employing the preferred embodiments and are subject to change without departing from such principles.

Therefore, this invention includes all modifications encompassed within the spirit of the following claims.

What is claimed is:

1. An improved keratome having a body movable with respect to a suction ring assembly during a stroke of the body, the improvement comprising:
wherein said body includes spring structure constructed and arranged to resiliently contact a portion of the suction ring assembly at an end of the stroke of the body.

2. The keratome of claim 1, wherein said body includes a gear plate carrying gears for causing movement of the body via gear interaction, said spring structure being associated with the gear plate.

3. The keratome of claim 2, wherein said spring structure is made integral with said gear plate and includes a cantilever having a hinged end and a free end opposite the hinged end such that the free end may flex about the hinged end upon contact with the portion of the suction ring assembly.

4. The keratome of claim 3, wherein said gear plate includes surfaces defining an open channel along a length of the gear plate at a bottom thereof, the free end of the cantilever extending into the channel at an end of the gear plate to define a resilient stop surface of the gear plate.

5. The keratome of claim 4, wherein the resilient stop surface is constructed and arranged to resiliently engage a stop surface of the suction ring assembly an end of the stroke of the body.

6. A keratome comprising:
a body configured to be moved during a stroke thereof with respect to a fixed member; and
spring structure associated with the body and being constructed and arranged to resiliently contact a portion of the fixed member at an end of the stroke of the body.

7. The keratome of claim 6, wherein said spring structure is made integral with said body and includes a cantilever having a hinged end and a free end opposite the hinged end such that the free end may flex about the hinged end.

8. The keratome of claim 7, wherein said body includes surfaces defining an open channel along a length of the body at a bottom thereof, the free end of the cantilever extending into the channel to at an end of the body to define a resilient stop surface of the body.

9. The keratome of claim 8, wherein the resilient stop surface is constructed and arranged to engage a stop surface of the fixed member at an end of the stroke of the body.

10. A keratome comprising:
a body; and
means for moving the body during a stroke thereof with respect to a suction ring assembly, the body including means for resiliently contacting a portion of the suction ring assembly at an end of the stroke of the body.

11. The keratome of claim 10, wherein the means for resiliently contacting the suction ring assembly comprises a spring structure made integral with the body

12. The keratome of claim 11, wherein the spring structure comprises a cantilever having a hinged end and a free end opposite the hinged end such that the free end may flex about the hinged end upon contact with the portion of the suction ring assembly.

13. The keratome of claim 12, wherein said body includes surfaces defining an open channel along a length of the body at a bottom thereof, the free end of the cantilever extending into the channel to at an end of the body to define a resilient stop surface of the body.

14. The keratome of claim 13, wherein the resilient stop surface is constructed and arranged to engage a stop surface of the suction ring assembly an end of the stroke of the body.

15. A keratome and suction ring assembly combination comprising:
a suction ring assembly constructed and arranged to be fixed to an eyeball, the suction ring including a stop surface, and
a keratome having a body movable with respect to a suction ring assembly during a stroke of the body, the body including spring structure constructed and arranged to resiliently contact the stop surface of the suction ring assembly at an end of the stroke of the body.

16. The combination of claim 15, wherein said spring structure is made integral with said body and includes a cantilever having a hinged end and a free end opposite the hinged end such that the free end may flex about the hinged end.

17. The combination of claim 16, wherein said body includes surfaces defining an open channel along a length of the body at a bottom thereof, a portion of the suction ring assembly being received in the channel during the stroke of the body, the free end of the cantilever extending into the channel at an end of the body to define a resilient stop surface of the body.

18. The combination of claim 17, wherein the resilient stop surface is constructed and arranged to engage the stop surface of the suction ring assembly an end of the stroke of the body.

19. A method of damping a force due to contact of a keratome with a fixed assembly at an end of a stroke of the keratome, the fixed assembly including a stop surface and the keratome having a body movable with respect to the fixed assembly, the body including a resilient stop surface, the method including:
moving the body with respect to the fixed assembly such that at the end of the stroke the resilient stop surface of the body engages the stop surface of the fixed assembly so as to dampen a contact force between the fixed assembly and the body.

20. The method of claim 19, wherein said resilient stop surface is defined by spring structure which is integral with said body, the spring structure including a cantilever having a hinged end and a free end opposite the hinged end such that the free end may flex about the hinged end.
